(19) United States
(12) Patent Application Publication
(10) Pub. No.: US 2009/0258028 A1
(43) Pub. Date: Oct. 15, 2009

(54) Methods Of Forming Coatings For Implantable Medical Devices For Controlled Release Of A Peptide And A Hydrophobic Drug

(75) Inventors: Thierry Glauser, Redwood City, CA (US); Syed F.A. Hossainy, Fremont, CA (US); Irina Astafieva, Palo Alto, CA (US); Florencia Lim, Union City, CA (US); Xinmin Xu, Libertyville, IL (US); Bozena Z. Maslanka, Aptos, CA (US); Mikael O. Trollsas, San Jose, CA (US); Michael H. Ngo, San Jose, CA (US)

Correspondence Address: SQUIRE, SANDERS & DEMPSEY LLP, 1 MARITIME PLAZA, SUITE 300, SAN FRANCISCO, CA, 94111, US

(73) Assignee: Abbott Cardiovascular Systems Inc., Santa Clara, CA (US)

(21) Appl. No.: 12/424,489

(22) Filed: Apr. 15, 2009

Related U.S. Application Data
(63) Continuation-in-part of application No. 11/447,551, filed on Jun. 5, 2006.
Application No. 11/447,551 is a continuation-in-part of application No. 12/165,521, filed on Jun. 30, 2008.

(51) Int. Cl. A61K 38/00; A61K 39/395

(52) U.S. Cl. 424/175.1; 514/2

(57) ABSTRACT

This invention is generally related to methods of forming coatings for implantable medical devices, such as drug delivery vascular stents. The methods are for forming coatings to control the release of a peptide such as RGD, and a hydrophobic drug. Both single layer and multiple layer coating constructs are encompassed in the various embodiments of the present invention.

### CROSS-REFERENCE TO RELATED APPLICATIONS

This application is a continuation-in-part of U.S. application Ser. No. 11/447,551 filed 5 Jun. 2006, and a continuation-in-part of U.S. application Ser. No. 12/165,521 filed 30 Jun. 2008, both of which are hereby incorporated by reference as if fully set forth, including any figures, herein.

### FIELD

This invention is generally related to methods of forming coatings for implantable medical devices.

### BACKGROUND

Percutaneous coronary intervention (PCI) is a procedure for treating heart disease. A catheter assembly having a balloon portion is introduced percutaneously into the cardiovascular system of a patient via the brachial or femoral artery. The catheter assembly is advanced through the coronary vasculature until the balloon portion is positioned across the occlusive lesion. Once in position across the lesion, the balloon is inflated to a predetermined size to radially compress the atherosclerotic plaque of the lesion to remodel the lumen wall. The balloon is then deflated to a smaller profile to allow the catheter to be withdrawn from the patient's vasculature.

Problems associated with the above procedure include formation of intimal flaps or torn arterial linings which can collapse and occlude the blood conduit after the balloon is deflated. Moreover, thrombosis and restenosis of the artery may develop over several months after the procedure, which may require another angioplasty procedure or a surgical by-pass operation. To reduce the partial or total occlusion of the artery by the collapse of the arterial lining and to reduce the chance of thrombosis or restenosis, a stent is implanted in the artery to keep the artery open.

Drug delivery stents have reduced the incidence of in-stent restenosis (ISR) after PCI (see, e.g., Serruys, P. W., et al., J. Am. Coll. Cardiol. 39:393-399 (2002)), which has plagued interventional cardiology for more than a decade. However, ISR still poses a significant problem given the large volume of coronary interventions and their expanding use. The pathophysiological mechanism of ISR involves interactions between the cellular and acellular elements of the vessel wall and the blood. Damage to the endothelium during PCI constitutes a major factor for the development of ISR (see, e.g., Kipshidze, N., et al., J. Am. Coll. Cardiol. 44:733-739 (2004)).

Advances in the technology of drug delivery devices, in particular stents, are a continuing goal in the field of interventional medicine. The current invention provides a significant addition to the field that involves methods of forming coatings for the controlled concurrent delivery of a peptide and a hydrophobic drug to a diseased situs in the vasculature of a patient.

### SUMMARY

The current invention is directed to methods of forming a coating on medical devices such that controlled release of both a peptide and/or a protein and a hydrophobic drug may be obtained.

Thus, an aspect of the invention is a method of fabricating a coating for a medical device that controls the release of both a hydrophobic drug and a peptide. The method comprises: providing an implantable medical device; providing a solvent; providing a semi-crystalline or amorphous polymer having a weight average molecular weight of not less than 50,000 Daltons, having a glass transition temperature, when plasticized with water under physiological conditions, of not more than 45° C., and having a solubility parameter between about 5 and about 25 (cal/cm³)^{1/2}, providing a peptide selected from the group consisting of RGD, cRGD, natriuretic peptide CNP, natriuretic peptide ANP, natriuretic peptide BNP, glycoprotein IIb/IIIb antagonists, Abciximax, anti-₃-integrin antibody F11, laminin derived SIKVAV, laminin derived YIGSR, KQAGDV, VAPG, and any combination thereof, and providing a hydrophobic drug. The method further comprises dissolving the peptide, the drug, and the polymer in the solvent to form a coating solution wherein the mass ratio of the peptide to the hydrophobic drug is from about 1:5 to about 5:1 and the mass ratio of the sum of the peptide and the hydrophobic drug to the polymer is from about 1:1 to about 1:7. The method further comprises disposing the coating solution over a surface of the implantable medical device and removing the solvent.

In an aspect of this invention, the hydrophobic drug provided has an aqueous solubility of not more than 10 mg/ml.

In an aspect of this invention, the hydrophobic drug provided has an aqueous solubility of not more than 5 mg/ml.

In an aspect of this invention, the hydrophobic drug provided has an aqueous solubility of not more than 1 mg/ml.

In an aspect of this invention, the thickness of the resulting coating layer is about 1 micron to about 10 microns.

In an aspect of this invention, disposing the solution over the implantable medical device comprises spraying the solution onto the surface of the device.

In an aspect of this invention, the mass ratio of the peptide to the polymer is about 1:1 to about 1:5.

In an aspect of this invention, the mass ratio of the sum of the peptide and hydrophobic drug to the polymer is about 1:3 to about 1:5.

In an aspect of this invention, the mass ratio of the sum of the peptide and hydrophobic drug to the polymer is about 1:1 to about 1:5.

In an aspect of this invention, the mass ratio of the peptide to the hydrophobic drug to the polymer is about 1:1:1: to about 1:1:5.

In an aspect of this invention, the peptide to hydrophobic drug mass ratio is about 2:1 to about 1:2.

In an aspect of this invention, the polymer, when plasticized with water under physiological conditions, has a glass transition temperature not greater than 42° C.

In an aspect of this invention, the polymer, when plasticized with water under physiological conditions, has a glass transition temperature not greater than 37° C.

In an aspect of this invention, the polymer is a copolymer of ε-caprolactone and at least one monomer that would form an aliphatic polyester.

In an aspect of this invention, the solubility parameter of the polymer is from about 6 (cal/cm³)^{1/2} to about 16 (cal/cm³)^{1/2}.

In an aspect of this invention, the polymer is a co-polymer of two or more monomers wherein at least one monomer has a solubility parameter of greater than or equal to 12.9 (cal/cm³)^{1/2} and at least one monomer has a solubility parameter that differs from that of the drug by not more than 2.5 (cal/cm³)^{1/2}.

In an aspect of this invention, the monomer(s) with a solubility parameter of greater than or equal to 12.9 (cal/cm³)^{1/2} comprise at least 25 mole % of the polymer and the monomer(s) with a solubility parameter that differs from that of the drug by not more than 2.5 (cal/cm³)^{1/2} comprise at least 25 mole % of the polymer.

In an aspect of this invention, the polymer comprises a hydrophilic block selected from the group consisting of poly(ethylene glycol), poly(vinyl pyrrolidone), poly(vinyl alcohol), poly(vinyl acetate), and combinations thereof.

In an aspect of this invention, the polymer comprises a poly(ester-amide) or an amphiphilic block copolymer.

In an aspect of this invention, the poly(ester-amide) has the formula:

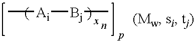

wherein i is an integer from 1 to 10, inclusive; j is an integer from 1 to 10, inclusive;
xₙ is an integer from 1 to 100, inclusive; p is an integer from 2 to about 9000;
M_{w} is from about 10,000 to about 1,000,000 Da; sᵢ is a number from 0 to 0.5, inclusive;
tⱼ is a number from 0 to 0.5, inclusive; with the proviso that Σᵢ sᵢ=Σⱼ tⱼ=0.5; Σᵢ sᵢ>0;
Σⱼ tⱼ>0; each Aᵢ has the chemical structure:

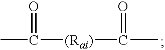

and each Bⱼ has the chemical structure

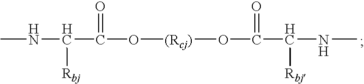

wherein: each R_{bj}, and R_{bj′} is independently selected from the group consisting of hydrogen and (C₁-C₄)alkyl, wherein: the alkyl group is optionally substituted with a moiety selected from the group consisting of —OH, —SH, —SeH, —C(O)OH, —NHC(NH)NH₂,

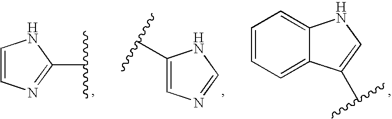

phenyl and

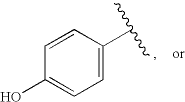

one or more of R_{bj} and R_{bj′} forms a bridge between the carbon to which it is attached and an adjacent nitrogen, the bridge comprising —CH₂CH₂CH₂—;
   each Rₐᵢ and each R_{cj} is independently selected from the group consisting of (C1-C12)alkyl, (C2-C12)alkenyl, (C3-C8)cycloalkyl, —(CH₂CH₂O)_{q}CH₂CH₂— wherein q is an integer from 1 to 10, inclusive,

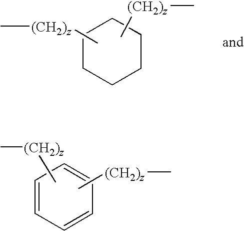

where z is 0, 1, or 2.

In an aspect of the invention, the polymer is a poly(ester-amide) polymer of the formula of the previous paragraph that is a random copolymer of constitutional units A₁-B₁, A₁-B₂, A₂-B₁ and A₂-B₂ or where t₁ is 0.125 to 0.375 and s₁ is 0.125 to 0.375, or A₁-B₁ and A₁-B₂ where t₁ is 0.125 to 0.375.

In an aspect of the invention, for the poly(ester-amide) polymer of the formula above p is an integer from 2 to 4500.

In an aspect of the invention, the polymer is a poly(ester-amide) polymer of the previous paragraph for which at least one of Rₐ₁, Rₐ₂ (if present), R_{c1}, or R_{c2} is selected from the group consisting of consisting of

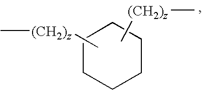

where z is 0, 1, or 2; and at least one of Rₐ₁, Rₐ₂ (if present), R_{c1}, or R_{c2} are —(CH₂)ₘ where m is an integer equal to or greater than 8.

In an aspect of the invention, for the poly(ester-amide) polymer, i=1 or 2, and j=2, and each of Rₐ₁ is selected from the group consisting of —(CH₂)₄—, —(CH₂)₅—, —(CH₂)₆—, —(CH₂)₇—, —(CH₂)₈, —(CH₂)₁₀—, —(CH₂)₁₁—, and —(CH₂)₁₂—; each of R_{b1}, R_{b1′}, R_{b2} and R_{b2′} are the same, and are selected from the group consisting of —(CH₂)—(CH(CH₃)₂), —CH₃), —CH(CH₃)₂, —(CH₂)₂—CO(NH₂), —CH(CH₃)—CH₂—CH₃, CH(OH)(CH₃), —CH₂—CO—(NH₂), —(CH₂)₄NH₃⁺, —(CH₂)₂—COO⁻, —(CH₂)₃NH—C(NH₂⁺)NH₂, —(CH₂)₂—S—(CH₃), and —(CH₂)—SH; R_{c1} is selected from the group consisting of —(CH₂)₄—, —(CH₂)₅—, —(CH₂)₆—, —(CH₂)₇—, —(CH₂)₈—, —(—CH₂CH₂O—)₁(CH₂)₂—, —(—CH₂CH₂O—)₂(CH₂)₂—, and —(—CH₂CH₂O—)₃(CH₂)₂—; R_{c2} is selected from the group consisting of

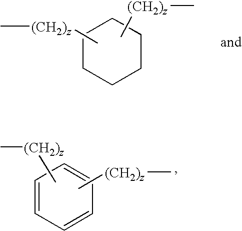

where z is 0, 1, or 2; and t₁ is 0.125 to 0.375.

In an aspect of the invention, the solvent is ethanol.

In an aspect of the invention, the solvent has a boiling point of 80° C. or less.

In an aspect of the invention, the drug has a solubility parameter equal to or lower than about 11.5 (cal/cm³).^{1/2}

In an aspect of the invention, the hydrophobic drug is selected from the group consisting of Biolimus A9, deforolimus, AP23572, tacrolimus, temsirolimus, pimecrolimus, zotarolimus, everolimus, 40-O-(3-hydroxypropyl)rapamycin, 40-O-[2-(2-hydroxy)ethoxy]ethyl-rapamycin, 40-O-tetrazole-rapamycin, 40-O-tetrazolylrapamycin, 40-epi-(N1-tetrazole)-rapamycin, paclitaxel, and combinations thereof.

In another aspect of the invention is a method of fabricating a coating for a medical device that controls the release of both a hydrophobic drug and a peptide that uses application of at least two coating formulations. The method comprises: providing an implantable medical device; providing a first solvent; providing a first polymer, a semi-crystalline or amorphous polymer, having an number average molecular weight of not less than 50,000 Daltons; having a glass transition temperature, when plasticized with water under physiological conditions, of not more than 45° C.; and having a solubility parameter between about 5 to about 25 (cal/cm³)^{1/2}; providing a peptide selected from the group consisting of RGD, cRGD, natriuretic peptide CNP, natriuretic peptide ANP, natriuretic peptide BNP, glycoprotein IIb/IIIb antagonists, Abciximax, anti-₃-integrin antibody F11, laminin derived SIKVAV, laminin derived YIGSR, KQAGDV, VAPG, and any combination thereof; providing a hydrophobic drug that is different from the peptide; dissolving the peptide and the first polymer, and optionally the hydrophobic drug, in the first solvent to form a first coating solution where the mass ratio of the peptide to polymer, or the sum of peptide and hydrophobic drug to polymer if the hydrophobic drug is added, is from 3:1 to 1:10; disposing the first coating solution over a surface of the implantable medical device; and removing the solvent. The method further includes optionally forming an optional intermediate coating layer by: providing an intermediate layer solvent, which may be the same as or different from the first solvent; dissolving the hydrophobic drug in the intermediate layer solvent to form an intermediate layer coating solution; and disposing the intermediate layer coating solution over a coated surface of the implantable medical device; and removing the solvent. The method further includes providing a second solvent, which may be the same as or different from either the first solvent and/or the optional intermediate layer solvent; providing a second semi-crystalline or amorphous polymer, having an average molecular weight of not less than 50,000 Daltons; having a glass transition temperature, when plasticized with water under physiological conditions, of not more than 45° C.; and having a solubility parameter that differs from the solubility parameter of the first polymer by not more than 10 (cal/cm³)^{1/2}; dissolving the second polymer, and optionally the hydrophobic drug, in the second solvent to form a second coating solution; wherein if the second coating solution comprises the hydrophobic drug, the mass ratio of the drug to polymer is from 1:1 to 1:5; disposing the second coating solution over a coated surface of the implantable medical device; and removing the solvent. At least one of the first, second, or optional intermediate coating solutions comprises the hydrophobic drug.

In an aspect of the invention, the hydrophobic drug included in at least one of the first, second, or optional intermediate coating solutions has an aqueous solubility of not more than 10 mg/ml.

In an aspect of the invention, the hydrophobic drug included in at least one of the first, second, or optional intermediate coating solutions has an aqueous solubility of not more than 5 mg/ml.

In an aspect of the invention, the hydrophobic drug included in at least one of the first, second, or optional intermediate coating solutions has an aqueous solubility of not more than 1 mg/ml.

In an aspect of the invention, disposing the first coating solution, the optional intermediate coating solution and the second coating solution comprises spraying the solution on the surface or a coated surface of the device.

In an aspect of the invention, the mass ratio of the sum of the peptide and hydrophobic drug to the first polymer or mass ratio of peptide to polymer if no hydrophobic drug is present is about 1:4 to about 1:8.

In an aspect of the invention, the second coating solution comprises the hydrophobic drug.

In an aspect of the invention, the first polymer, when plasticized with water, has a glass transition temperature not greater than 50° C.

In an aspect of the invention, the first polymer, when plasticized with water, has a glass transition temperature not greater than 42° C.

In an aspect of the invention, the first polymer, when plasticized with water, has a glass transition temperature not greater than 37° C.

In an aspect of the invention, the second polymer, when plasticized with water, has a glass transition temperature not greater than 42° C.

In an aspect of the invention, the second polymer, when plasticized with water, has a glass transition temperature not greater than 37° C.

In an aspect of the invention, the first polymer has a number average molecular weight of about 60,000 to about 150,000 Daltons.

In an aspect of the invention, at least one of the first or second polymers has a weight average molecular weight of at least 50,000 Daltons.

In an aspect of the invention, the first polymer is an amphiphilic block copolymer comprising a polar block.

In an aspect of the invention, the polar block of the first polymer is selected from the group consisting of poly(urethane), poly(HEMA-block-MMA), poly(HEMA-block-HPMA), poly(HPMA-GFLG), poly(butyl methacrylate-co-ethylene glycol acrylate) (poly(BMA-block-PEGA)) poly(MOEMA-block-HEMA), and any combination thereof.

In an aspect of the invention, the polar block of the first polymer comprises no less than 25 mole % of the polymer and no more than 75 mole % of the polymer.

In an aspect of this invention, the solubility parameter of the at least one of the first and second polymers is from about 6 (cal/cm³)^{1/2} to about 16 (cal/cm³)^{1/2}.

In an aspect of the invention, the second polymer has a solubility parameter about equal to or less than 12 (cal/cm³)^{1/2}.

In an aspect of the invention, the first polymer is a block copolymer formed from the reaction of two or more monomers wherein at least one monomer or one has a solubility parameter of greater than or equal to 12.9 (cal/cm³)^{1/2} and at least one monomer has a solubility parameter that differs from the drug by not more than 2.5 (cal/cm³)^{1/2}.

In an aspect of the invention, the first polymer is a copolymer formed from the reaction of two or more monomers wherein at least one monomer or one has a solubility parameter of greater than or equal to 12.9 (cal/cm³)^{1/2} and at least one monomer has a solubility parameter that differs from the drug by not more than 2.5 (cal/cm³)^{1/2}.

In an aspect of the invention, for the first polymer, the monomers with a solubility parameter of greater than or equal to 12.9 (cal/cm³)^{1/2} comprise at least 25 mole % of the polymer and the monomers with a solubility parameter that differs from the drug by not more than 2.5 (cal/cm³)^{1/2} comprise at least 25 mole % of the polymer.

In an aspect of the invention, at least one of the first solvent, second solvent, and optional intermediate layer solvent has a boiling point of 80° C. or less.

In an aspect of the invention, the first solvent, second solvent, and the optional intermediate layer solvent have a boiling point of 80° C. or less.

In an aspect of the invention, at least one of the first solvent, second solvent, and the optional intermediate layer solvent is ethanol.

In an aspect of the invention, the hydrophobic drug included in at least one of the first, intermediate, or second coating solutions has a solubility parameter equal to or lower than about 11.5 (cal/cm³).^{1/2}

### BRIEF DESCRIPTION OF THE DRAWINGS

FIG. 1 is a schematic of a generalized multilayer coating construct of the present invention.

FIGS. 2A, 2B, and 2C are graphs illustrating concurrent release of a hydrophobic drug and a peptide from an implantable medical device of this invention.

FIGS. 3 and 4 are graphs showing the individual release profiles of cRGD protein and everolimus from implantable medical devices of this invention having drug reservoir layers comprising two different polymers.

### DETAILED DESCRIPTION

As used herein, unless specified otherwise, any words of approximation such as without limitation, “about,” “essentially,” “substantially” and the like mean that the element so modified need not be exactly what is described, but can vary from the description by as much as ±15% without exceeding the scope of this invention.

As used herein, any ranges presented are inclusive of the end-points. For example, “a temperature between 10° C. and 30° C.” or “a temperature from 10° C. to 30° C.” includes 10° C. and 30° C., as well as any temperature in between.

As used herein, an “implantable medical device” refers to any type of device that is totally or partly introduced, surgically or medically, into a patient's body or by medical intervention into a natural orifice, and which is intended to remain there after the procedure. The duration of implantation may be essentially permanent, i.e., intended to remain in place for the remaining lifespan of the patient; until the device biodegrades; or until it is physically removed. Examples of implantable medical devices include, without limitation, implantable cardiac pacemakers and defibrillators; leads and electrodes for the preceding; implantable organ stimulators such as nerve, bladder, sphincter and diaphragm stimulators, cochlear implants; prostheses, vascular grafts, self-expandable stents, balloon-expandable stents, stent-grafts, grafts, artificial heart valves, cerebrospinal fluid shunts, and intrauterine devices. An implantable medical device specifically designed and intended solely for the localized delivery of a drug is within the scope of this invention.

As used herein with respect to an implantable medical device, “device body,” refers to an implantable medical device in a fully formed utilitarian state having an outer surface to which no coating or layer of material different from that of which the device is manufactured has been applied. By “outer surface” is meant any surface however spatially oriented that is in contact with bodily tissue or fluids. A common example of a “device body” is a BMS, i.e., a bare metal stent, which, as the name implies, is a fully-formed usable stent that has not been coated with a layer of any material different from the metal of which it is made on any surface that is in contact with bodily tissue or fluids. Of course, device body refers not only to BMSs but to any uncoated device regardless of what it is made of.

A type of implantable medical device is a “stent.” A stent refers generally to any device used to hold tissue in place in a patient's body. Particularly useful stents, however, are those used for the maintenance of the patency of a vessel in a patient's body when the vessel is narrowed or closed due to diseases or disorders including, without limitation, tumors (m, for example, bile ducts, the esophagus, the trachea/bronchi, etc.), benign pancreatic disease, coronary artery disease, carotid artery disease and peripheral arterial disease such as atherosclerosis, restenosis and vulnerable plaque.

As used herein, “drug” refers to any substance that, when administered in a therapeutically effective amount to a patient suffering from a disease, has a therapeutic beneficial effect on the health and well-being of the patient. A therapeutic beneficial effect on the health and well-being of a patient includes, but it not limited to: (1) curing the disease or condition; (2) slowing the progress of the disease or condition; (3) causing the disease or condition to retrogress; or, (4) alleviating one or more symptoms of the disease or condition.

As used herein, a drug also includes any substance that when administered to a patient, known to be or suspected of being particularly susceptible to a disease, in a prophylactically effective amount, has a prophylactic beneficial effect on the health and well-being of the patient. A prophylactic beneficial effect on the health and well-being of a patient includes, but is not limited to: (1) preventing or delaying on-set of the disease or condition in the first place; (2) maintaining a disease or condition at a retrogressed level once such level has been achieved by a therapeutically effective amount of a substance, which may be the same as or different from the substance used in a prophylactically effective amount; or, (3) preventing or delaying recurrence of the disease or condition after a course of treatment with a therapeutically effective amount of a substance, which may be the same as or different from the substance used in a prophylactically effective amount, has concluded.

As used herein, “drug” also encompasses salts, esters, amides, prodrugs, active metabolites, analogs, and the like of those drugs specifically mentioned.

As used herein, a “peptide” refers to a molecule comprising from 2 to 49 amino acids. Chains of 50 amino acids or more are referred to as “proteins.”

As used herein, a “polymer” is a molecule made up of the repetition of a simpler unit, herein referred to as a repeat unit. The repeat units themselves can be the product of the reactions of other compounds. A polymer may comprise one or more types of repeat units. As used herein, the term polymer refers to a molecule comprising 2 or more repeat units. A “monomer” is compound which may be reacted to form a polymer, or part of a polymer, but is not itself the repetition of a simpler unit. As a non-limiting example, CH₂═CH₂ or ethylene is reacted to form polyethylene, such as CH₃(CH₂CH₂)₅₀₀CH₃, for which the repeat unit is —CH₂—CH₂— and for which ethylene CH₂═CH₂ is the monomer. As used herein, an “oligomer” is a polymer of fewer than 20 repeat units.

As used herein, “copolymer” refers to a polymer including more than one type of repeat unit.

A polymer herein may be a regular alternating polymer, a random alternating polymer, a regular block polymer, a random block polymer or a purely random polymer unless expressly noted otherwise.

As used herein, a “poly(ester-amide)” refers to a polymer that has in its backbone structure a multiplicity of ester and amide bonds.

As used herein “polyester” will refer to a substance “that meets the definition of polymer and whose polymer molecules contain at least two carboxylic acid ester linkages, at least one of which links internal monomer units together.” (Adapted from 40 CFR 750.250(b)) An aliphatic polyester is a polyester without any cyclic groups whether aromatic rings or cycloalkyl cyclic groups.

As used herein, the terms “biodegradable,” “bioerodable,” and “bioabsorbable,” are used interchangeably to refer to polymers, coatings, coating layers, and materials that are capable of being completely, or substantially completely, degraded, eroded and/or absorbed over time when exposed to physiological conditions. Conversely, a “biostable” polymer, coating, coating layer, or material is not biodegradable.

The “glass transition temperature,” T_{g}, is the temperature at which an amorphous polymer or an amorphous segment of a block copolymer or a changes mechanical properties from elastic to brittle. The measured T_{g} of a given polymer is dependent on the heating rate and is influenced by the thermal history, and potentially pressure history, of the polymer, as well as potentially the pressure at which the measurement is made. The chemical structure of the polymer heavily influences the glass transition by affecting chain mobility.

Some polymers have additional transitions below T_{g}. T_{g} may also be referred to as the α-transition and is associated with the segmental motion of the backbone of the polymer. Motion of the side chains or parts of the backbone may show transitions below T_{g} and these transitions are often referred to as β or γ transitions (the first below T_{g} is the β transition, etc.).

Plasticization of a polymer refers to the addition of a lower molecular weight material to a polymer, which results in a lower T_{g} of the blend of the polymer and the plasticizer.

As used herein, a “semi-crystalline polymer” is one which has regions that exhibit the characteristics of a crystalline polymer such as crystallites, that is regions where the chains are folded and arranged in a regular structure, and regions that are amorphous.

As used herein, a “polymer molecular weight” refers to a weight average molecular weight as determined by gel permeation chromatography utilizing a polystyrene standard, unless expressly stated otherwise.

As used herein, the “water absorption” of a polymer is determined as the mass uptake of water at a pH of about 6 to about 7.5 by the polymer at 37° C. and standard atmospheric pressure, or about standard atmospheric pressure.

As used herein, a layer or film “disposed over” a surface refers to such layer or film being deposited directly or indirectly over at least a portion of the surface. Direct depositing means that the coating layer is applied directly to the surface of the substrate. Indirect depositing means that the coating layer is applied to an intervening layer that has been deposited directly or indirectly over the substrate. A coating layer is supported by a surface of the substrate, whether the coating layer is deposited directly, or indirectly, onto the surface of the substrate. The term “coating layer” will be used refer to the material of the same, or substantially the same, composition that is deposited in the same unit operation (essentially at the same time) despite the fact that multiple passes or applications of material may be required to obtain a layer of appropriate thickness. The terms “layer” and “coating layer” will be used interchangeably and refer to a layer, film, or coating layer as described in this paragraph. Unless the context clearly indicates otherwise, a reference to a layer or a coating layer refers to a layer of material that covers all, or substantially all, of a surface whether deposited directly or indirectly. Unless expressed stated otherwise the coating does not chemically bind to the substrate. As used herein, the term “coating” refers to one or more layers disposed over a substrate as described in this paragraph.

As used herein, “above” a surface or layer is defined as farther from the substrate measured along an axis normal to a surface, or over a surface or layer, but not necessarily in contact with the surface or layer.

As used herein, “below” a surface or layer is defined as closer to the substrate measured along an axis normal to a surface, or under a surface or layer, but not necessarily in contact with the surface or layer.

As used herein, “solvent” is defined as a substance capable of dissolving or dispersing one or more substances, fully or partially, to form a uniform dispersion or solution at a selected temperature and pressure. A solvent can refer to one compound, or a mixture of compounds. A solvent may be a fluid.

As used herein, a “coating formulation” refers to the compounds disposed over a device surface, either directly and indirectly, and which are intended to remain on the surface to form the coating layer. Thus, if coating materials are dissolved or dispersed in a solvent and the solution or dispersion is disposed over a surface, the solvent is not part of the “coating formulation” as most of it is removed to form the coating.

As used herein, the term “solubility parameter,” δ, refers to the cohesive energy density of a substance. The solubility parameter of a substance is calculated as follows:

δ=(ΔE/V)^{1/2}

where δ is the solubility parameter, (cal/cm³)^{1/2};

ΔE is the energy of vaporization, cal/mole; and

V is the molar volume, cm³/mole.

As used herein, a “hydrophobic drug” is one for which the solubility in water at 37° C., standard atmospheric pressure and pH in the range of 6.0 to 7.5 is about equal to or less than 20 mg/ml.

As used herein, “cumulative drug release” refers to the total amount of drug released from the drug delivery system over a given period of time expressed as a percent of the total drug content contained in the drug delivery system. A non-limiting example of a drug delivery system is a stent having a coating including drug.

As used herein, “substantially released,” refers to a cumulative release of a drug of about 80% or more.

As used herein, “sustained release” refers to a drug delivery profile in which the drug is released over an extended period of time.

As used herein, “burst release” refers to the uncontrolled release of a drug from a drug delivery system over a relatively short time compared to the desired release duration.

As used herein, the “duration of release” refers to the time period starting with initial drug release and ending at the time of 80% cumulative release.

As used herein, a coating, or coating layer, that “controls the release” of a drug refers to a coating for which the cumulative release of the drug is less than 90% in 24 hours but is at least 5% in 72 hours.

As used herein, any measurement of drug release, for example without limitation, release rate, cumulative release, or substantially released, refers to an in-vitro measurement of drug release utilizing scintillation vials of 20 ml (or another vial or container) that are shaken on an Orbit Environ Shaker at about 175 rpm (or substantially equivalent equipment) with porcine serum with optionally sodium azide added (such as, without limitation, at 0.3% w/v) at a temperature of 37° C. as the dissolution media. The coated substrate is submerged in scintillation vials containing 20 ml of Porcine Serum. At each time point, a number of coated substrates are removed and saved for extraction analysis, and the porcine serum solutions are discarded. The drugs remaining in the coating is determined by an appropriate assay such as, without limitation, HPLC. The volume of solution, the size of vial, and the time-points for removal of the substrate for later assay, may vary depending upon the coated substrate being tested.

In the discussion that follows, reference may be made to the control of release of a peptide, or a particular peptide, RGD. However, the various embodiments of the present invention are not so limited and encompass other peptides as well as proteins.

Various embodiments of the present invention encompass methods of forming a coating on an implantable medical device that controls the release of a peptide and a hydrophobic drug. The design of the coating involves: selection of the coating construct, that is a single layer or multiple layers; the selection of one or more polymers and/or other rate-controlling components of the coating in light of the characteristics of the particular peptide and hydrophobic drug; the selection of peptide to drug ratio, and peptide plus drug to polymer ratio; the selection of the method of coating application and any solvents, if required, for application; and thickness of the coating, or of the various layers comprising the coating.

A polymer of the present invention is biocompatible and may be biodegradable or biostable. Polymer blends may be used and may include combinations of biodegradable and biostable polymers. The polymer may be cross-linked to form a network, although in presently preferred embodiments the methods do not involve cross-linking the polymers. The hydrophobic drug and/or peptide may be chemically bound to one or more polymers in the coating but in presently preferred embodiments they are not so bound.

### Single Layer Constructs

In some embodiments, a coating that controls the release of a peptide and a hydrophobic drug may be formed as a single coating layer such that the release of both the peptide and the hydrophobic drug is controlled by one coating layer. If the hydrophobic drug and the peptide are included in one coating layer, the polymer chosen for the coating layer may be compatible with both the peptide and drug and soluble in a solvent in which both the peptide and the drug are soluble. The polymer may have a molecular weight of at least 50,000 Daltons, or as presently preferred at least about 100,000 Daltons. Although there is no theoretical upper limit on the molecular weight, the coatings are typically applied from solution, and thus higher molecular weight polymers may exhibit a viscosity or other characteristic of very high molecular weight polymers that curtail their applicability. Such will be readily recognizable to those skilled in the art.

A polymer herein may be amorphous or semi-crystalline. If semi-crystalline, the crystallinity should be about 40% or less, preferably about 30% or less, and more preferably at present about 20% or less. The polymer, or a block of the polymer if the polymer is a block copolymer, may have a glass transition temperature of about 45° C. or less when plasticized under physiological conditions, that is, water at a pH of about 6.5 to 7.5 and a temperature of about 37° C. In some embodiments, the polymer may exhibit additional transitions below the glass transition, such as a β or γ transitions. The additional transition may be at least 15° C. lower than the glass transition, but should be no more than 60° C. lower.

The solubility parameter for the polymer, or the weight average solubility parameter for the polymers in a blend, may be about 5 to about 25 (cal/cm³)^{1/2}, preferably about 8.3 to about 13.3 (cal/cm³)^{1/2}, more preferably about 8.8 to about 12.8 (cal/cm³)^{1/2} and as most preferred at present about 9.3 to about 12.3 (cal/cm³)^{1/2}. The polymer, or the polymer blend, may have both hydrophobic and hydrophilic characteristics. In some embodiments the polymer, or the blend of polymers, may absorb at least 3% by mass but no more than 12% by mass water. The polymer, or one of the polymers of a blend, may be a copolymer, whether random or block copolymer, for which at least one repeat unit is hydrophobic, or one polymer of the blend is hydrophobic, that is it has a solubility parameter for the purposes of this invention of 11.5 (cal/cm³)^{1/2} or less and at least one other repeat unit or polymer in the blend, the more hydrophilic unit, has a solubility parameter of about 12.9 (cal/cm³)^{1/2} or higher, and in some embodiments 14.0 (cal/cm³)^{1/2} or higher. In some embodiments, the hydrophobic polymer has a solubility parameter that differs from that of the drug by not more than about 3.0 (cal/cm³)^{1/2}, preferably at present not more than about 2.5 (cal/cm³)^{1/2}. The polymer, or one of the polymers of a blend, may have a repeat unit derived from vinyl pyrrolidone, ethylene glycol, vinyl alcohol, vinyl acetate, and/or a repeat unit which contains acid groups. The polymer may be a copolymer including choline, a phospholipid, a semi-synthetic phosphoryl choline such as cardiolipin or sphingosine, a natural phospholipid including phosphoryl choline, phosphoryl serine, phosphoryl inositol, di-phosphoryl glycerol, zwitterionic phosphoryl ethanolamine, and combinations thereof. The copolymers are described in co-pending patent application Ser. No. 10/807,362 filed on Mar. 22, 2004, and the copolymers so described are hereby incorporated by reference, herein.

Other polymers that may be utilized include poly(ester-amide) polymers. One group of poly(ester-amide) polymers that may be used in the various embodiments of this invention have the generic formula:

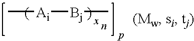

wherein the repeat units are represented by Aᵢ-Bⱼ where the Aᵢ and Bⱼ react to form the repeat unit represented by Aᵢ-Bⱼ. The Aᵢ groups are derived from diacids, and the Bⱼ groups are derived from diamino esters. Thus, each Aᵢ has the chemical structure:

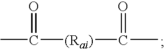

and each Bⱼ has the chemical structure

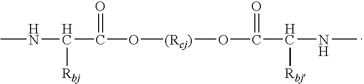

The repeat units themselves may be the product of the reactions of other compounds. For example, without limitation, a Bⱼ group above can comprise the reaction of an amino acid,

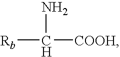

with a diol, HO—(R_{c})—OH, to give a diamino ester,

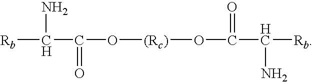

While a poly(ester-amide) that may be used in the various methods of this invention may be constructed from any amino acid, particularly useful amino acids are the so-called essential amino acids of which there currently 20: alanine, arginine, asparagine, aspartic acid, cysteine, glutamic acid, glutamine, glycine, histidine, isoleucine, leucine, lysine, methionine, phenyl alanine, proline, serine, threonine, tryptophan, tyrosine and valine. More recently selenoadenine has been found to be incorporated into a number of proteins and is also included. In naturally-occurring biological proteins, these amino acids appear as the l-enantiomeric isomers but for the purposes of this invention they may be used as their l- or d-enantiomers or as racemic mixtures.

Each Aᵢ and Bⱼ represent one or more different groups derived from diacids or derived from diamino esters, respectively, which may react to form the repeat units, where i represents the i^{th} type of Aᵢ group, and j represents the j^{th} type of Bⱼ group. Each polymer may have from 1 to 10 Aᵢ groups, and from 1 to 10 Bⱼ groups. A particular polymer may have fewer than the maximum, or 10, different Aᵢ groups. Thus if i=3, there is an A₁, A₂ and A₃ group. Similarly a particular polymer may have fewer different Bⱼ groups than the maximum, 10. Therefore, if j=2, there is a B₁ and a B₂ group. There must be at least one Aᵢ group, and at least one Bⱼ group.

The subscripts Xₙ is an integer which represents the number of different possible types of Aᵢ-Bⱼ repeat units in a polymer chain, and p is an integer which represents the average total number of repeat units in an average polymer chain. Thus, each xₙ is an integer from about 0 to about 100. The number of different xₙ groups is a function of the number of different Aᵢ groups and different Bⱼ groups as there is an xₙ for each Aᵢ-Bⱼ group. For example if there are two Aᵢ groups and three Bⱼ groups, there will be six possible Aᵢ-Bⱼ groups (A₁-B₁, A₁-B₂, A₁-B₃, A₂-B₁, A₂-B₂, A₂-B₃), and six xₙ's (x₁, X₂, X₃, X₄, X₅, X₆). The average number of repeat units in a chain, p, is an integer from 2 to about 20,000.

The polymer represented by the above formula may be a random, alternating, random block or alternating block polymer. The term “-/-” used between repeat units, for example, [-(A₁-B₂)-/-(A₁-B₂)-], means that the Aᵢ-Bⱼ group may be attached to or reacted with another Aᵢ-Bⱼ group wherein both Aᵢ and Bⱼ are the same or one or both of Aᵢ and Bⱼ differ.

In the above formula, each of the sᵢ, and tⱼ, represent the average mole fraction of each of the Aᵢ, and Bⱼ, respectively, which react to form the repeat units which form the polymer. Each of the sᵢ, tⱼ, is a number between 0 and 0.5, inclusive and subject to the constraint that Σᵢ sᵢ=Σⱼ tⱼ=0.5 where each summation of sᵢ is from 1 to the number of different Aᵢ groups (maximum of 10), and each summation of tⱼ is from 1 to the number of different Bⱼ groups (maximum of 10). The mole fraction and the number of repeat units are obviously related and it is understood that the designation of one will affect the other.

As used herein, “alkyl” refers to a straight or branched chain fully saturated (no double or triple bonds) hydrocarbon (carbon and hydrogen only) group. Examples of alkyl groups include, but are not limited to, methyl, ethyl, propyl, isopropyl, butyl, isobutyl, tertiary butyl, pentyl, hexyl, ethenyl, propenyl, butenyl, cyclopropyl, cyclobutyl, cyclopentyl, and cyclohexyl. As used herein, “alkyl” includes “alkylene” groups, which refer to straight or branched fully saturated hydrocarbon groups having two rather than one open valences for bonding to other groups. Examples of alkylene groups include, but are not limited to methylene, —CH₂—, ethylene, —CH₂CH₂—, propylene, —CH₂CH₂CH₂—, n-butylene, —CH₂CH₂CH₂CH₂—, sec-butylene, —CH₂CH₂CH(CH₃)— and the like.

As used herein, “Cm to Cn,” wherein m and n are integers refers to the number of possible carbon atoms in the indicated group. That is, the group can contain from “m” to “n”, inclusive, carbon atoms. An alkyl group in a PEA polymer as outlined above may comprise from 1 to 20 carbon atoms that is m may be 1 and n may be 20. The alkyl group may be linear or branched. For example without limitation, a “C1 to C4 alkyl” group refers to all alkyl groups having from 1 to 4 carbons, that is, CH₃—, CH₃CH₂—, CH₃CH₂CH₂—, CH₃CH(CH₃)—, CH₃CH₂CH₂CH₂—, CH₃CH₂CH(CH₃)— and (CH₃)₃CH—.

As use herein, a “cycloalkyl” group refers to an alkyl group in which the end carbon atoms of the alkyl chain are covalently bonded to one another. The numbers “m” to “n” then refer to the number of carbon atoms in the ring so formed. Thus for instance, a (C3-C8)cycloalkyl group refers to a three, four, five, six, seven or eight member ring, that is, cyclopropane, cyclobutane, cyclopentane, cyclohexane, cycloheptane and cyclooctane.

As used herein,

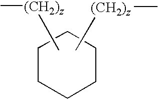

represents a cyclohexane group with a —CH₂— or a —CH₂—CH₂— alkylene group optionally attached at any two locations on the ring, i.e., at the 1 & 2, 1& 3, or 1 & 4 positions. Alternatively, if z=0, the ring may be attached to the other atoms in the molecule at the 1 & 2, 1 & 3, or 1 & 4 positions. The substituent groups, or the bonds with other molecules, may be either cis or trans to one another.

As used herein, “alkenyl” refers to an alkyl group that contains one or more double bonds.

As used herein, “alkynl” refers to an alkyl group that contains one or more triple bonds.

The PEA polymers used in the methods of this invention may have at least one Rₐᵢ and/or R_{ci} that provides some chain stiffness that is groups with some bonds that limit free rotation. Such groups include, but are not limited to, cycloalkyls such as, without limitation, cyclohexane (obtained from reacting the cyclohexane diol), or another cycloalkyl of 5-8 carbons, or one or more aromatic rings. In some embodiments, the PEA polymer may contain as one or more Rₐᵢ and/or R_{ci} groups that include multiple rings joined by an alkylene or poly(alkyl ether) group such as —CH₂CH₂O—CH₂CH₂— (more generally for such embodiments, (—(CH₂)ₐ—O—)_{b}—(CH₂)ₐ— where a=2, 3, or 4 and b=1 to 10). The mole fraction of the sum of all of such repeat units with chain stiffing groups may be about 0.25 to about 0.75. The PEA polymer may have a Rₐᵢ group that has some elastic properties, that is with bonds that allow free rotation. Such groups include but are not limited to C4-C16 straight chain alkyls, or C6 to C12 alkyl ethers (or poly(alkyl ethers)) such as polyethylene glycol or polypropylene oxide. The mole fraction of the sum of all of such repeat units with elastic groups may be about 0.25 to about 0.75. It is presently preferred that alkyl groups of C3 or lower are not present, and if C4 alkylene groups are present, the sum of the mole fractions of the repeat units with C4 groups is about 0.4 or less.

The PEA polymer may have an R_{bj} group that is selected from, or all R_{bj} groups that are selected from, the group consisting of —(CH₂)—(CH(CH₃)₂), —CH₃), —CH(CH₃)₂, —(CH₂)₂—CO(NH₂), —CH(CH₃)—CH₂—CH₃, CH(OH)(CH₃), —CH₂—CO—(NH₂), —(CH₂)₄NH₃⁺, —(CH₂)₂—COO⁻, —(CH₂)₃NH—C(NH₂⁺)NH₂, —(CH₂)₂—S—(CH₃), and —(CH₂)—SH. In some embodiments, all R_{bj} groups are the same.

In some embodiments, the polymers may be subject to the proviso that at least one Rₐᵢ or at least one R_{cj} is selected from the group consisting of

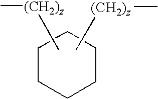

where z is 0, 1, or 2. The Rₐᵢ is part of the Aᵢ group, and the R_{cj} is part of the Bⱼ group.

Some embodiments of the present invention use poly(ester-amide) random copolymers of two repeat units, A₁-B₁ and A₁-B₂, in which s₁ is about 0.5, t₁ is between 0.125 and 0.375, t₂ is 0.5-t₁, and p is an integer from 2 to about 9000. Thus x=2, or there is an x₁ and an x₂ group. Rₐ₁ of group A₁ is selected from the group consisting of —(CH₂)₄—, —(CH₂)₅—, —(CH₂)₆—, —(CH₂)₇—, —(CH₂)₈—, —(CH₂)₉—, —(CH₂)₁₀—, —(CH₂)₁₁—, —(CH₂)₁₂—, —(—CH₂CH₂O—)₁(CH₂)₂—, —(—CH₂CH₂O—)₂(CH₂)₂—, —(—CH₂CH₂O—)₃(CH₂)₂—, and —(—CH₂CH₂O—)₄(CH₂)₂—, R_{b1}, R_{b1′}, R_{b2} and R_{b2′} are all the same, and are selected from the group consisting of —(CH₂)—(CH(CH₃)₂), —CH(CH₃)₂, —CH(CH₃)—CH₂—CH₃, —(CH₂)₂—S—(CH₃), —(CH₂)—SH and —(CH₃), R_{c1} is selected from the group consisting of —(CH₂)₄—, —(CH₂)₅—, —(CH₂)₆—, —(CH₂)₇—, —(CH₂)₈—, —(CH₂)₉—, CH₂)₁₀—, —(CH₂)₁₁—, —(CH₂)₁₂—, —(—CH₂CH₂O—)₁(CH₂)₂—, —(—CH₂CH₂O—)₂(CH₂)₂—, —(—CH₂CH₂O—)₃(CH₂)₂—, and —(—CH₂CH₂O—)₄(CH₂)₂—, and R_{c2} is selected from the group consisting of

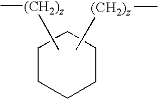

where z is 0, 1, or 2. In some embodiments, R_{c2} is selected from

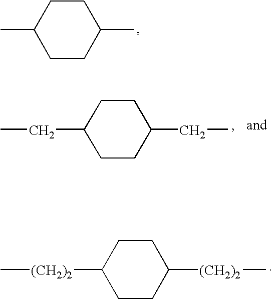

Some embodiments of the present invention use poly(ester-amide) polymer which is a random co-polymer of the following formula [-(A₁-B₁)-/-(A₁-B₂)-]ₚ (M_{w}, s₁, t₁, t₂):

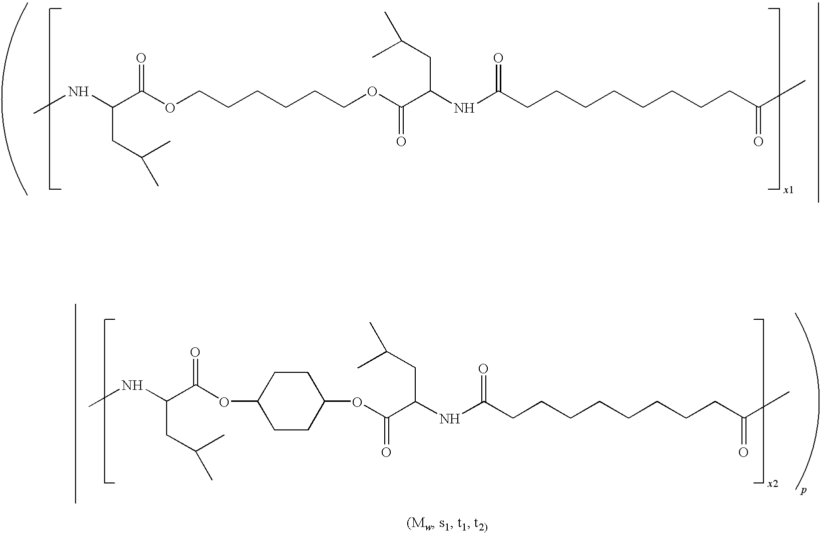

in which s₁ is 0.5, and each of t₁ and t₂ are about 0.25, and thus the mole fractions of the two repeat units, X1 and X2, are each about 0.5, and p is the total number of X1 and X2 units on average, per polymer chain and ranges from 20 to about 9000. The two groups do not necessarily alternate regularly as this is a random copolymer, and there are multiple X1 and X2 groups per polymer chain. There may be large variations in the length of the polymer chains.

In other embodiments, other polymers may be used including polymers of monomers which would form an aliphatic polyester, copolymerized with ε-caprolactone. In a presently preferred embodiment, the mole fraction of repeat units derived from ε-caprolactone is about 10% to about 25%. Non-limiting examples include random copolymers of 50%:25%:25% D,L-lactide/glycolic acid/ε-caprolactone, 60%:15%:25% L-lactide/glycolic acid/ε-caprolactone, 60%:15%:25% D,L-lactide/glycolic acid/ε-caprolactone, 65%:15%:2% D,L-lactide/glycolic acid/ε-caprolactone, and 78%:22% D,L-lactide/ε-caprolactone.

Other polymers include acrylate polymers and methacrylate polymers with pendant styrene units or co-polymerized with styrene. The aromatic ring styrene provides both stiffness and hydrophobicity, and the acrylate or methacrylate backbone provides flexibility. The mol % styrene monomer is a function of the tacticity of the styrene with higher mol % styrene possible for atactic polystyrene which does not form crystals. A non-limiting example is a styrene-isobutylene-styrene block copolymer in which the Tg of the isobutylene block is below 37° C. or about 37° C.

In other single-layer construct embodiments there is not one polymer, but a blend of polymers. The polymers of the blend may be soluble in a common solvent with the peptide and the drug. The polymers may be amorphous or semi-crystalline. With a polymer blend, a crystalline polymer may be used provided that the weight % of crystalline polymer in the blend is not more than 20%, preferably not more than 15%, or most preferably at present not more than 10%. At least 40%, at least 50%, or preferably at present at least 60% of the polymer in the polymer blend is amorphous and has a glass transition temperature of about 45° C. or under when plasticized under physiological conditions.

A polymer blend may include one or more block copolymers having hydrophobic and hydrophilic blocks to enhance the miscibility of other hydrophilic and hydrophobic polymers in the blend. The block copolymer acts similarly to a surfactant by partitioning to the interface between the hydrophobic and hydrophilic polymer. The relative ratios of hydrophobic polymer to hydrophilic polymer and the relative ratio of the blocks in the block copolymer, as well as the quantity of the block copolymer control the resulting morphology of the blend. The morphology may be one in which one of the hydrophobic or hydrophilic phases forms a separate dispersed phase in the other analogous to an emulsion, or the two phases may be co-continuous, i.e., each forms a continuous network.

The ratio of the peptide to the hydrophobic drug, and the ratio of the sum of the peptide and hydrophobic drug to the polymer is another factor influencing release rate of the drug and peptide. The choice of a higher amount of polymer generally results in a slower release, while too small an amount may lead to too rapid release of the drug and/or peptide. Furthermore, polymer content impacts the physical and mechanical integrity of the coating layer. In embodiments in which the peptide and hydrophobic drug are included in one coating layer without a rate-limiting layer, the mass ratio of the peptide to the hydrophobic drug may be in the range of about 1:0.1 to about 1:10, or about 1:0.2 to about 1:5, or as presently preferred, about 1:0.25 to about 1:4.

With respect to the drug to polymer ratio, the ratio of the peptide to the ratio of polymer may vary from about 1:0.1 to about 1:10, or about 1:0.2 to about 1:5, or as presently preferred, about 1:0.5 to about 3:1. In some embodiments, the ratio of the sum of the peptide and hydrophobic drug to the polymer may be about 1:1 to about 1:7.

The coating may be disposed over the surface of the device by any number of methods including, but not limited to electrostatic coating, plasma deposition, dipping, brushing, or spraying. In a presently preferred embodiment a coating solution is sprayed onto the device. The spraying may be carried out by atomizing the solution and spraying it onto the device surface while rotating and translating the device underneath the spray nozzles following by rotation and translation under a flow of gas, such as air or nitrogen. Multiple passes underneath the spray nozzles and the gas may be required to obtain a desired layer thickness. Subsequently, the device may be heated to remove residual solvent. Thus, in general, a coating layer is the result of the application of the multiple passes in one process before the device is subjected to an operation for the removal of residual solvent, or before application of a different coating solution. Materials from one layer may incidentally diffuse or migrate into another layer. In some cases, material may be extracted, or partially extracted, from a layer or layers that have already been applied during the application of subsequent layers.

With respect to coating layer thickness, it may be in the range of about 0.5 and about 9 μm, or about 0.5 and about 7 μm, or as presently preferred, about 2 and about 7 μm.

### Multi-Layer Constructs

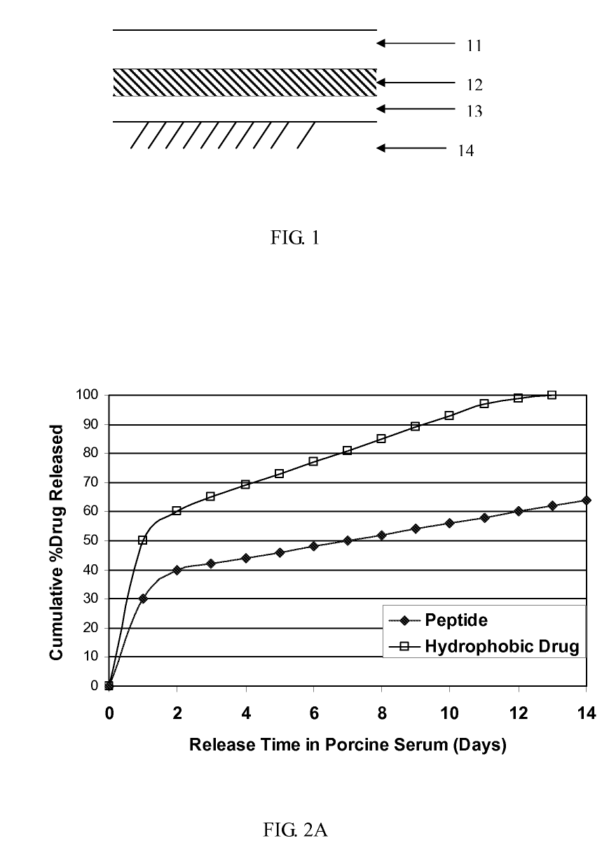

In other embodiments, the coating construct has more than one layer, or in other words, the coating results from the disposing over the device at least two different coating formulations at different times. In these embodiments, two coating layers are used to control the release of both a hydrophobic drug and a peptide. The second layer may be a rate-controlling layer for at least one of the hydrophobic drug and peptide. Some embodiments utilize a first coating layer including both the hydrophobic drug and the peptide, followed by the formation of a second layer above the first layer that controls, or helps to control, the release of the peptide and/or hydrophobic drug. In other embodiments, a first coating layer is formed including the peptide, with a second coating layer above the first, and with an intermediate coating layer including the hydrophobic drug formed between the first and second layers. In other embodiments, the first coating layer includes the peptide and the second coating layer, formed above the first coating layer, includes the hydrophobic drug. FIG. 1 depicts the generalized multi-layer construct including a first layer 13 on top of the substrate or device surface 14, an optional intermediate layer 12, and the second layer 11. Thus, as used herein with respect to the multilayer embodiments, the term “second layer” will be used to refer to a layer including polymer above the first layer similar to that depicted in FIG. 1, regardless of whether or not there is an intermediate layer.

In those multi-layer embodiments in which the hydrophobic drug and the peptide are included in one layer, the polymer chosen for the first coating layer may be compatible with both the peptide and drug, but less compatibility may work for a multi-layer construct that would not work for the single layer construct as there is at least one additional layer (the second layer) to control release. The polymer chosen may be soluble in a solvent in which both the peptide and the drug are soluble. The polymer may have a number average molecular weight of at least 50,000 Daltons, and in some embodiments, the number average molecular weight is from about 60,000 Daltons to about 150,000 Daltons. In some embodiments, the weight average molecular weight is at least 50,000 Daltons.

The polymer may be amorphous or semi-crystalline. If semi-crystalline, the crystallinity may be about 40% or less, preferably about 30% or less, or most preferably at present about 20% or less. The polymer may have a glass transition temperature of about 50° C. or less, preferably about 45° C. or less when plasticized under physiological conditions. In some embodiments, the polymer may have a β or γ transition, which may be at least 15° C. lower than the glass transition but not more than 60° C. lower.

The polymer may have a solubility parameter that is about 5 to about 25 (cal/cm³)^{1/2}, preferably about 8.3 to about 13.3 (cal/cm³)^{1/2}, more preferably about 8.8 to about 12.8 (cal/cm³)^{1/2}, or presently most preferred about 9.3 to about 12.3 (cal/cm³)^{1/2}. In some embodiments, the polymer may have both hydrophobic and hydrophilic characteristics. The polymer may absorb at least 3% but not more than 12% water. The polymer may be a copolymer, whether random or block copolymer, for which at least one repeat unit is or one polymer in a blend may be hydrophobic, that has a solubility parameter of about 11.5 (cal/cm³)^{1/2} or less, and at least one other repeat unit or one other polymer in the blend, the more hydrophilic unit or polymer, has a solubility parameter of about 12.9 (cal/cm³)^{1/2}, and in some embodiments greater or in some embodiments about 14.0 (cal/cm³)^{1/2} or greater. In some embodiments, the hydrophobic unit in the polymer may have a solubility parameter that differs from that of the drug by not more than 10.0 (cal/cm³)^{1/2}, preferably not more than 5.0 (cal/cm³)^{1/2}, more preferably at present not more than 3.0 (cal/cm³)^{1/2}, and most preferably at present not more than 1.5 (cal/cm³)^{1/2}. The polymer may have a repeat unit derived from vinyl pyrrolidone, ethylene glycol, vinyl alcohol, vinyl acetate, and/or a repeat unit which contains acid groups. The polymer may be a copolymer including a choline or phospholipid moieties or one polymer in a blend choline or phospholipid moieties, such as those described previously herein.

In those multi-layer construct embodiments in which the hydrophobic drug is included in a layer different from the peptide containing layer, the polymer of the first coating layer may have similar properties in terms of crystallinity and glass transition and other transitions, to those polymers described in the previous three paragraphs. The polymer may be soluble in any solvent in which the peptide is also soluble, but does not need to be soluble in a solvent for the hydrophobic drug.

In the various multi-layer construct embodiments, whether both peptide and the hydrophobic drug included in the first coating layer, or only the peptide included in the first coating layer, the polymer may be a polymer with a polar block or segment. Non-limiting examples include poly(urethane) such as without limitation BIOSPAN™ (Polymer Technology Group of Berkeley, Calif.), a segmented poly(urethane urea), poly(HEMA-block-MMA), poly(HEMA-block-HPMA), poly(HPMA-GFLG), poly(butyl methacrylate-co-ethylene glycol acrylate) (poly(BMA-block-PEGA)) or poly(MOEMA-block-HEMA). MOEMA stands for methoxyethyl methacrylate, HEMA for hydroxylethyl methacrylate, MMA for methyl methacrylate and HPMA for hydroxylpropyl methacrylate. HPMA-GFLG is HPMA terminated with the peptide sequence GFLG (glycine-pheylaniline-leucine-glycine) which is used as a linker. Presently preferred are di-block and triblock copolymers including up to about 15% HEMA such as without limitation, poly(HEMA-block-MMA), poly(HEMA-block-HPMA), or poly(MOEMA-block-HEMA), or up to 15% PEGA such as without limitation poly(BMA-block-PEGA).

In some embodiments, a poly(ester amide) polymer such as those described in the section on single layer constructs may be used for the first coating layer which may include the peptide, or both the peptide and the hydrophobic drug.

For the multi-layer construct embodiments, the polymer used in the second layer may amorphous or semi-crystalline. If semi-crystalline, the crystallinity may be about 60% or less, preferably about 40% or less, more preferably at present about 30% or less. The polymer be a hydrophobic polymer that a solubility parameter of about 11.5 (cal/cm³)^{1/2} or less. In some embodiments, the polymer may include some hydrophilic segments or repeat units with a solubility parameter of about 12.9 (cal/cm³)^{1/2} or higher, and preferably at present about 14.0 (cal/cm³)^{1/2} or higher. In some embodiments, the polymer of the second layer differs by not more than 10.0 (cal/cm³)^{1/2}, preferably not more than 5.0 (cal/cm³)^{1/2}, more preferably not more than 3.0 (cal/cm³)^{1/2}, and most preferably at present not more than 1.5 (cal/cm³)^{1/2}, than the solubility parameter of the polymer of the first layer, or from the weight averaged solubility parameters of the several polymers if multiple polymers are included in the layer. The polymer may have a glass transition temperature of about 42° C. or less when plasticized with water under physiological conditions. In some embodiments, the polymer may absorb at least 3% water but not more than 12% water. The polymer used in the second layer may be soluble in a solvent that does not dissolve or extract the components of the first layer, or an intermediate layer, if present.

Non-limiting examples of polymers for the second layer include polymers or copolymers of fluorinated olefin (e.g., SOLEF™ polymers from Solvay Fluoropolymers, Inc. of Houston, Tex.) such as but not limited to poly(vinylidene fluoride-co-hexafluoropropene) (SOLEF™ 21508). In some embodiments, the hydrophobic polymer can also include a small percentage of units derived from a small percentage of a hydrophilic monomer. Some examples of such polymers include, but are not limited to, poly(MOEMA-HEMA) and poly(MOEMA-PEGA) with low percentage of HEMA or PEGA (e.g., <10 mol %). In these polymers, the hydrophobic portion of the polymer can control the release of a hydrophobic drug, such as without limitation, everolimus, while the small percentage units derived from a hydrophilic monomer can allow for slow release of a peptide, such as cRGD. Poly(MOEMA) may be dissolved in a solvent of dimethyl acetamide and methanol at a volume:volume ratio of 1:2 to 1:4, or alternatively, in dimethyal acetamide: pentane at similar ratios.

In the multi-layer construct embodiments, the mass ratio of the sum of the peptide and the hydrophobic drug (or the peptide only for those embodiments without hydrophobic drug in the first coating layer) to the polymer in the first coating layer may be about 3:1 to about 1:10, preferably about 1:4 to about 1:8, and most preferably at present about 1:6.

In those embodiments in which the second coating layer includes a hydrophobic drug, the ratio of drug to polymer may be about 1:1 to about 1:5. In some embodiments, the hydrophobic drug is applied as an intermediate coating layer above the first coating layer and below the second coating layer. The intermediate layer may be all, or essentially all, hydrophobic drug or may include a binder, a polymer, or another component up to about 20% by mass. In a presently preferred embodiment the intermediate layer is at least 90% by mass hydrophobic drug.

The thickness of the first coating layer may be in the range of about 0.5 to about 9 μm, preferably about 0.5 to about 7 μm, most preferably at present about 2 to about 7 μm. The second layer thickness may be from about 0.5 to about 5 μm, preferably at present about 1 to about 3 μm.

The coating layers may be disposed over the surface of the device by any number of methods, but spraying from solution is a presently preferred method, as previously described. For the application of multiple coating layers, it is presently preferred to apply the first layer, remove residual solvent, and then apply the second layer. In those embodiments including an intermediate layer comprising primarily the hydrophobic drug, the intermediate layer may be applied after the first coating layer has been applied, but prior to treatment to remove residual solvent or after the treatment to remove residual solvent.

### All Coating Constructs

All embodiments of the present invention, whether the hydrophobic drug and the peptide are included in one layer or in different layers, encompass optional additional coating layers. In all embodiments, a primer layer which is the layer in direct contact with the device surface and which typically serves as an adhesive layer or intermediary layer between the device surface and the subsequently applied layer, may be included. There may be any number of layers below the first coating layer that includes either the peptide and drug or only the peptide.

All embodiments may optionally include a top-coat layer that is on the outer surface and above all of the other layers. If the topcoat layer is not intended to be a rate-controlling or rate-limiting layer, it may be formulated to quickly dissolve and/or erode. There may be any number of other layers between, above, or below the previously mentioned layers.

In all embodiments, any layer may include one or more additional drugs other than the hydrophobic drug and/or the peptide. The various embodiments may include more than one peptide, and/or more than one hydrophobic drug in any layer.

The coating formulation for one or more of the layers may include substances other than polymer, peptide, hydrophobic drugs, and/or additional drug(s) such as, without limitation, fillers, binders, carriers, plasticizers, stabilizers, and other additives. In some embodiments, there are no, or essentially no, other substances in the coating layer (or in the coating formulation) other than the polymer, drugs (including the hydrophobic drug and any additional drugs) and/or peptide. In other embodiments, the coating layer may include up to about 5%, 10%, 15%, or 20% of other substances. In some embodiments, the coating layer is entirely, or essentially entirely, composed of polymer, hydrophobic drug, and/or peptide. Residual solvents used in coating layer formation may be present in any coating layers.

### Release Profiles

Sustained release of a peptide, such as RGD, may be used to recruit endothelial cells or endothelial progenitor cells to the surface. To recruit endothelial cells or endothelial progenitor cells to the surface, the RGD peptide may diffuse through the polymer coating, through a layer of absorbed proteins and cells (acute phase after implantation), and through the neo-intima (long-term phase) to the lumen surface. There may be an amount of peptide sufficient to recruit endothelial cells or endothelial progenitor cells to the surface, and the peptide may be delivered over an extended period of time.

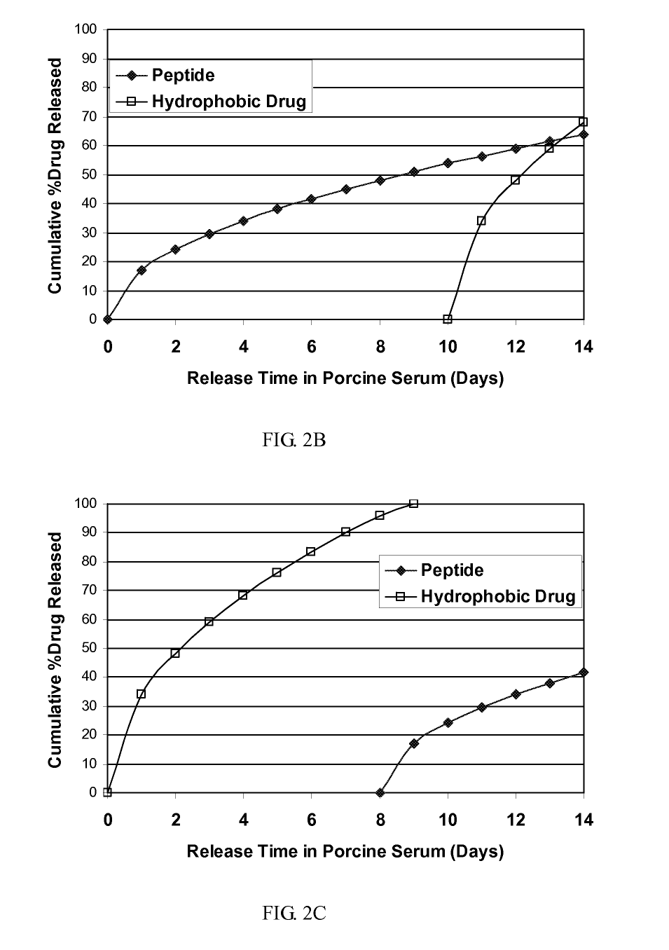

The release of the drug and the peptide from a coating resulting from the methods of the present invention may be at least partially concurrent, that is that at least 10% of the total release of one occurs during a time period when the other is also being released. As presently preferred, there is a significant overlap, 30%, or 40%, or more preferably 50% or more of the release of one during a time period in which the other is being released. FIGS. 2A, 2B, and 2C are non-limiting examples of desirable concurrent release profiles. FIGS. 2A, 2B, and 2C do not represent actual experimental data but are included for illustrative purposes. Concurrent release may be obtained by appropriate selection of polymers, the ratios of peptide to drug, and the ratio of the sum of drug and peptide to polymer, and type of coating construct as set forth herein.

Some embodiments result in a coating for which the peptide may be released over a time period from 1 month to 3 months, and/or the hydrophobic drug may be released over about 1 week to about 1 month. In some embodiments, the sustained release may be zero-order, that is a constant rate over time, or substantially zero-order, or zero-order or substantially zero-order for some portion of the cumulative release, such as without limitation, 40% to 75% of the cumulative release. A zero-order, or substantially zero-order, release may be obtained by use of a multi-layer coating construct in which the second layer is the rate controlling layer for the drug and/or peptide in the layer below. In some embodiments, the rate of release may decrease with time, or follow a square root of time release pattern. A square-root-of-time release pattern may be obtained by using a single layer construct in which the drug and/or peptide are homogeneously, or essentially homogeneously, dispersed or distributed.

In some embodiments, the hydrophobic drug may be substantially released at a time when the cumulative release of the peptide is less than about 80%, while in other embodiments, the peptide may be substantially released at a time when the cumulative release of the hydrophobic drug is less than about 80%. Such release profiles may be obtained if either the drug and/or peptide is in the second layer and the other in the first. Alternatively, a coating composition in which one of the drug or peptide has a higher diffusion coefficient in the coating layer than the other may also be used.

In some embodiments, there may be a delay in the release of the peptide, followed by sustained release of the peptide. In some embodiments, there may be a delay in the release of the hydrophobic drug followed by a sustained release of the hydrophobic drug. A delayed release may be obtained in those embodiments with a second layer devoid of the peptide and/or hydrophobic drug due to the time lag during which the peptide and/or hydrophobic drug diffuses through the second layer in the coating. In those embodiments in which the release of either of the drugs is delayed, there may be some overlap in the release profiles (about 10% or more than 10% of the release of one occurs during a time period when the other is also being released). In either case there may be a delay in release followed by zero-order, or nearly zero-order, sustained release.

In some embodiments, there is a burst release of the peptide and/or hydrophobic drug followed by a sustained release, or a square-root-of-time release, or substantially square-root-of-time release. The release rate profile of the cRGD peptide with a burst will match the mechanistic temporal need for activation of an endothelial progenitor cell capture process. The long term release of the cRGD peptide at low doses can maintain the recruiting of EPCs and continue to affect the surrounding endothelial cells and smooth muscle cells. A burst release may be about 20% or less, about 20% to 30%, about 30% to about 40%, or about 40% to about 50% of the total drug released within 10% of the total duration of release. In some embodiments, a burst release may be about 20% or less, about 20% to 30%, about 30% to about 40%, or about 40% to about 50% of the total drug released within the first 24 hours if the duration of release is a week or more, or within the first 72 hours, if the duration of release is three weeks or more. A burst release may be obtained by utilizing a multilayer construct with the some fraction of the peptide and/or hydrophobic drug (the compound for which a burst release is desired) in the second layer of the coating, or alternatively, included in a layer that quickly dissolves or erodes and is above the second layer or above the rate controlling layer for that compound. A burst release may also be obtained by using a high drug to polymer ratio such as about 1:1, about 1:2, or about 1:3 in a single layer construct, or in the second layer of a multiple layer construct. For the peptide, a burst release may be obtained by using a more hydrophilic polymer for the peptide containing layer.

In some embodiments, the cumulative release of the peptide may range from about 5% to about 50% at 24 hours, and about 10% to about 95% at 7 days, preferably from about 5% to about 25% at 24 hours and about 10% to about 40% at 7 days, and more preferably at present from about 8% to about 15% at 24 hours, and about 12% to 25% at 7 days. In some embodiments, the cumulative release of the hydrophobic drug may range from about 5% to about 50% at 24 hours, and about 10% to about 95% at 7 days, preferably from about 10% to about 35% at 24 hours, and more preferably about 25% to about 75% at 7 days, preferably at present about 15% to about 30% at 24 hours, and about 45% to about 70% at 7 days.

### Peptides

The peptides that may be used in the various embodiments of the present invention include, but are not limited to, RGD, cRGD, and similar peptides. RGD is the polypeptide Arg-Gly-Asp (RGD) that has been demonstrated to be a bioactive factor for human endothelial cell attachment and therefore will be expected to exhibit prohealing characteristics. Prohealing refers to a substance that is biocompatible and that aids in the amelioration of inflammation, and/or promotes healing. The RGD sequence can be found in numerous proteins and extra-cellular matrix, as well as in short peptides whether they are linear, cyclic, free or linked. In addition to RGD itself, RGD peptide or cyclic RGD peptide (cRGD), synthetic cyclic RGD (cRGD) mimetics, and small molecules binding to other adhesion receptors differentially expressed on the endothelial cells, are within the scope of this invention. The cRGD or RGD mimetics described herein includes any peptides or peptide mimetics that result from the modification of the cyclic Arg-Gly-Asp peptide. The modification can be on the pendant groups and/or on the backbone of the peptide.

Other peptides similar in size to RGD or cRGD may also be used.

### Hydrophobic Drugs

Hydrophobic compounds typically have a low solubility parameter when compared to water. In some embodiments, a drug sufficiently hydrophobic to be used in the various methods of the present invention may have a solubility parameter of about 11.5 (cal/cm³)^{1/2} or lower.

In some embodiments, the hydrophobic drug utilized may be one that has a molecular weight in the range of 200 Da to 2000 Da, 500 Da to 1500 Da, or preferably at present 800 Da to 1100 Da.

One class of drugs that contains many hydrophobic drugs and which are presently particularly useful are anti-proliferative drugs. The term “anti-proliferative” as used herein, refers to a drug that works to block the proliferative phase of acute cellular rejection. Examples of anti-proliferative drugs include rapamycin (sirolimus), Biolimus A9 (Biosensors International, Singapore), deforolimus, AP23572 (Ariad Pharmaceuticals), tacrolimus, temsirolimus, pimecrolimus, zotarolimus (ABT-578), 40-O-(2-hydroxy)ethyl-rapamycin (everolimus), 40-O-(3-hydroxypropyl)rapamycin, 40-O-[2-(2-hydroxy)ethoxy]ethyl-rapamycin, 40-O-tetrazole-rapamycin, 40-O-tetrazolylrapamycin, and 40-epi-(N1-tetrazole)-rapamycin. The anti-proliferatives described herein are generally hydrophobic.

Any drugs having anti-proliferative effects can be used in the present invention. The anti-proliferative drug can be a natural proteineous agent such as a cytotoxin or a synthetic molecule. Other drugs included in the various embodiments of the present invention include, without limitation, anti-proliferative substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I₁, actinomycin X₁, and actinomycin C₁), all taxoids such as taxols, docetaxel, and paclitaxel, paclitaxel derivatives, all olimus drugs, FKBP-12 mediated mTOR inhibitors, and perfenidone, prodrugs thereof, co-drugs thereof, and combinations thereof.

Other potential drugs include, without limitation, estradiol, 17-beta-estradiol, nitric oxide donors, super oxide dismutases, super oxide dismutases mimics, 4-amino-2,2,6,6-tetramethylpiperidine-1-oxyl (4-amino-TEMPO), dexamethasone, γ-hiridun, clobetasol, dexamethasone acetate, mometasone, imatinib mesylate, midostaurin, feno fibrate, feno fibric acid, and prodrugs thereof, co-drugs thereof, and combinations thereof.

### Other Drugs

Drugs other than the peptide described above and the hydrophobic drug described above may be used in the various methods of the present invention. Examples of suitable drugs, which may also be classified as hydrophobic drugs and/or peptides, but are not necessarily so classified, include, but are not limited to, synthetic inorganic and organic compounds, proteins and peptides, polysaccharides and other sugars, lipids, and DNA and RNA nucleic acid sequences having therapeutic and/or prophylactic activities. Nucleic acid sequences include genes, antisense molecules that bind to complementary DNA to inhibit transcription, and ribozymes. Some other examples of other drugs include antibodies, receptor ligands such as the nuclear receptor ligands estradiol and the retinoids, enzymes, adhesion peptides, blood clotting factors, inhibitors or clot dissolving drugs such as streptokinase and tissue plasminogen activator, antigens for immunization, hormones and growth factors, oligonucleotides such as antisense oligonucleotides, ribozymes and retroviral vectors for use in gene therapy, and genetically engineered endothelial cells. Other drugs include heparin, fragments and derivatives of heparin, glycosamino glycan (GAG), GAG derivatives, alpha-interferon, and thiazolidinediones (glitazones). The drugs could be designed, e.g., to inhibit the activity of vascular smooth muscle cells. They could be directed at inhibiting abnormal or inappropriate migration and/or proliferation of smooth muscle cells to inhibit restenosis.

Examples of drugs that may be used in the various embodiments of the present invention include, without limitation, anti-restenosis, pro- or anti-proliferative, anti-inflammatory, anti-neoplastic, antimitotic, anti-platelet, anticoagulant, antifibrin, antithrombin, cytostatic, antibiotic, anti-enzymatic, anti-metabolic, angiogenic, cytoprotective, angiotensin converting enzyme (ACE) inhibiting, angiotensin II receptor antagonizing and/or cardioprotective drugs.

The antiproliferative drugs mentioned above also include, without limitation, angiopeptin, and fibroblast growth factor (FGF) antagonists.

Examples of anti-inflammatory drugs include both steroidal and non-steroidal (NSAID) anti-inflammatories such as, without limitation, clobetasol, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone, dexamethasone dipropionate, dexamethasone acetate, dexmethasone phosphate, momentasone, cortisone, cortisone acetate, hydrocortisone, prednisone, prednisone acetate, betamethasone, betamethasone acetate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus and pimecrolimus.

Alternatively, the anti-inflammatory drug can be a biological inhibitor of pro-inflammatory signaling molecules. Anti-inflammatory drugs include antibodies to such biological inflammatory signaling molecules.

Examples of antineoplastics and antimitotics include, without limitation, paclitaxel, docetaxel, methotrexate, azathioprine, vincristine, vinblastine, fluorouracil, doxorubicin hydrochloride and mitomycin.

Examples of anti-platelet, anticoagulant, antifibrin, and antithrombin drugs include, without limitation, heparin, sodium heparin, low molecular weight heparins, heparinoids, hirudin, argatroban, forskolin, vapiprost, prostacyclin, prostacyclin dextran, D-phe-pro-arg-chloromethylketone, dipyridamole, glycoprotein IIb/IIIa platelet membrane receptor antagonist antibody, recombinant hirudin and thrombin, thrombin inhibitors such as ANGIOMAX® (bivalirudin), calcium channel blockers such as nifedipine, colchicine, fish oil (omega 3-fatty acid), histamine antagonists, lovastatin, monoclonal antibodies such as those specific for Platelet-Derived Growth Factor (PDGF) receptors, nitroprusside, phosphodiesterase inhibitors, prostaglandin inhibitors, suramin, serotonin blockers, steroids, thioprotease inhibitors, triazolopyrimidine, nitric oxide or nitric oxide donors, super oxide dismutases, super oxide dismutase mimetic and 4-amino-2,2,6,6-tetramethylpiperidine-1-oxyl (4-amino-TEMPO).

Examples of ACE inhibitors include, without limitation, quinapril, perindopril, ramipril, captopril, benazepril, trandolapril, fosinopril, lisinopril, moexipril and enalapril.

Examples of angiogensin II receptor antagonists include, without limitation, irbesartan and losartan.

Other drugs include anti-infectives such as antiviral drugs; analgesics and analgesic combinations; anorexics; antihelmintics; antiarthritics, antiasthmatic drugs; anticonvulsants; antidepressants; antidiuretic drugs; antidiarrheals; antihistamines; antimigrain preparations; antinauseants; antiparkinsonism drugs; antipruritics; antipsychotics; antipyretics; antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary vasodilators; peripheral and cerebral vasodilators; central nervous system stimulants; cough and cold preparations, including decongestants; hypnotics; immunosuppressives; muscle relaxants; parasympatholytics; psychostimulants; sedatives; tranquilizers; naturally derived or genetically engineered lipoproteins; and restenoic reducing drugs.

Some drugs may fall into more than one of the above mentioned categories.

### EXAMPLES

The examples presented in this section are provided by way of illustration of the current invention only and are not intended nor are they to be construed as limiting the scope of this invention in any manner whatsoever. Each of the examples the follows relates to the coating of 3 mm×12 mm VISION (Abbott Cardiovascular Systems Inc.) stent, which has a coatable surface area of 0.5556 cm².

### Example 1

All stents were cleaned by being sonicated in isopropyl alcohol, followed by an argon plasma treatment. No primer layer was applied to the stents. Application of a coating layer on the stents was accomplished by spraying the stents with a solution of everolimus (Novartis): cRGD (Bachem, H-Gly-Pen-Gly-Arg-Gly-Asp-Ser): poly(ester-amide) at a 1:1:3 mass ratio in ethanol (anhydrous, 99.5+%, absolute ethanol). The weight % polymer in solution was 2%. The objective drug loading for each stent was 58 μg for each of the everolimus and cRGD. The poly(ester-amide) polymer used was that illustrated in below:

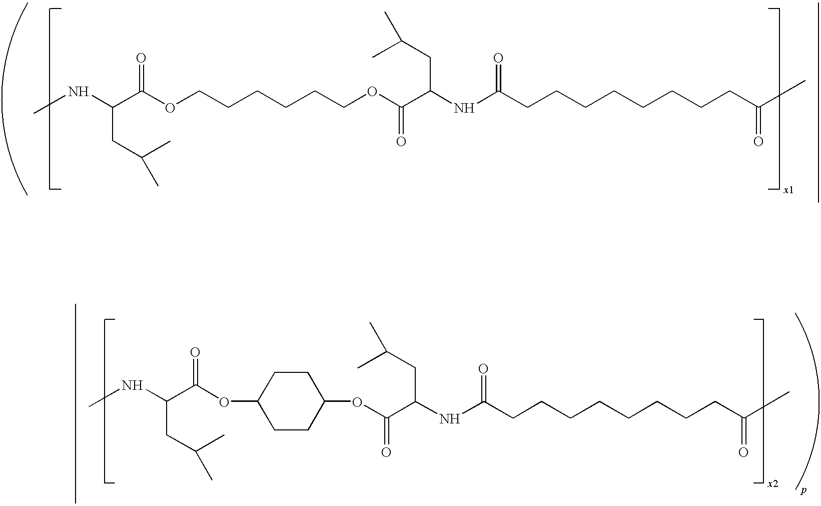

The polymer above is referred to as PEA-40, where the subscripts X1 and X2 indicate the two repeat units, and the p indicates multiple units. The PEA-40 utilized was approximately a 50:50 random copolymer of the two repeat units (s₁=0.5, and t₁=t₂=0.25), and had a weight-average molecular weight of in the range of about 100-120 KDa. The poly(ester-amide) polymer was manufactured by standard methods. The poly(ester-amide) polymer was purified and precipitated several times, and there were no detectable levels, or essentially no detectable levels, of residual reactants, solvents or catalysts.

The spraying operation was carried out with a custom made spray coater equipped with a spray nozzle, a drying nozzle, and a means to rotate and translate the stent under the nozzles with the processing parameters outlined in Table 1. Subsequent to coating, all stents were baked in a forced air convection oven at 50° C. for 60 minutes. More than one pass under the spray nozzle was required to obtain the target weight of coating layer on the stent. The coating layer thickness was about 5-6 μm. After heat treatment of the coating, the stents were crimped onto 3.0×12 mm Xience V catheters, placed into coil to protect the catheter, and then sealed in Argon filled foil pouches. These stents were sterilized by electron beam sterilization by one pass through the electron beam at 25 KGy.

TABLE 1
Spray Processing Parameters for Coating
Spray Head

| | |
|---|---|
| Spray nozzle air cap, | 0.028″ round |
| Spray nozzle temperature, ° C. | No heat, ambient |
| Atom pres (non-activated), psi | 15 ± 2.5 |
| Spray nozzle to mandrel dist, mm | 11 ± 1 |
| Solution flow rate, ml/hour or ml/min | 0.05 + 0.03 ml/min |

Heat Nozzle

| | |
|---|---|
| Temperature at stent site, ° C. | 62 ± 5 |
| Air Pressure, psi | 20 ± 2 |
| Spray nozzle to mandrel distance, psi | 11 ± 1 |

Coating Recipe(s)

| | |
|---|---|
| Spray time, seconds | 30 ± 15 |
| Dry time, seconds | 10 |

Flow Rate and Coating Weight

| | |
|---|---|
| Target Flow Rate (ref.), μg/pass (μg | 18 |
| solids per pass) | |

Cumulative release of both the everolimus and the cRGD peptide over 7 days was determined using an Orbit Environ Shaker. Each of nine stents were submerged in a scintillation vial containing 20 ml of Porcine Serum. At each time point, three stents were taken out and saved for extraction analysis and porcine serum solutions were discarded. The following parameter were employed
Agitation: 175 rpm
Temperature: 37° C.
Release Medium: Porcine Serum with 0.3% (w/v) Sodium Azide
Time points: day 1, day 3, day 7
Media volume: 20 ml

The remaining cRGD and Everolimus were extracted and analyzed by HPLC.

The percent of the cRGD and the everolimus remaining in the stent based upon the objective loading of 58 μg/stent, and a sample size of N=3 stents.

### Example 2

Stents were coated as in Example 1 with the exception that a different coating solution was utilized. The stents were sprayed with a solution of everolimus: cRGD: poly(ester-amide) at a 1:1:7 mass ratio in ethanol. The weight % solids in solution was 2%. The objective drug loading for each stent was 58 μg for each of the everolimus and cRGD. The poly(ester-amide) polymer was that illustrated below:

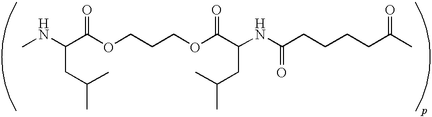

The above poly(ester-amide) polymer is also referred to as PEA-11. PEA-11 has only one repeat unit, and the subscript p indicates multiples of this unit. Similar to Example 1 described above, the polymer was manufactured by standard methods and purified. The poly(ester amide) utilized had a weight-average molecular weight of about 100-120 KDa. The coating layer thickness was about 5-6 μm. After sterilization, the stents were tested for cumulative drug release as described in Example 1.

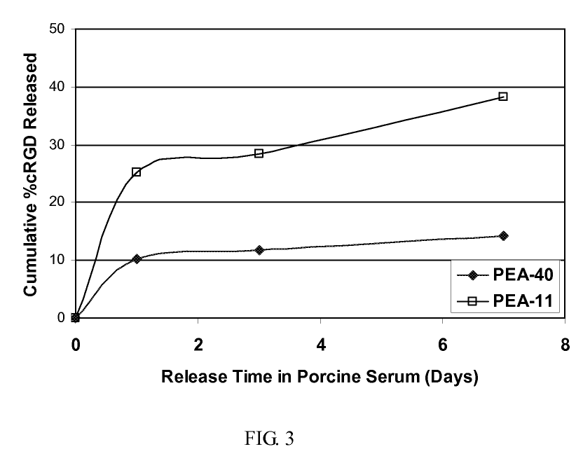

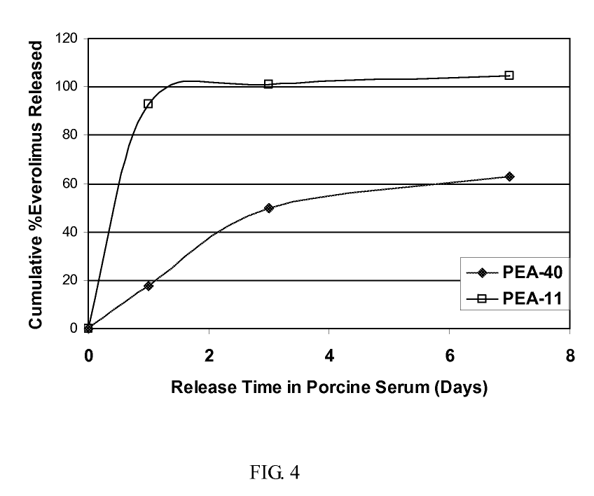

FIGS. 3 and 4 illustrate the release profiles for the cRGD protein and everolimus over 7 days for the two coating layers, that is the coating layer made with PEA-11 (Example 2) and the coating layer made with PEA-40 (Example 1). As illustrated in FIG. 4, the release of everolimus from the PEA-11 poly(ester-amide) coating layer is not beyond about 24 hours for the drug to polymer ratio used in Example 2. In contrast, FIGS. 3 and 4 illustrate that the coating layer including PEA-40 provides for a longer release duration of both everolimus and the cRGD peptide at the drug to polymer ratio used.

While particular embodiments of the present invention have been shown and described, it will be obvious to those skilled in the art that changes and modifications can be made without departing from this invention in its broader aspects. The various aspects of the invention may be used in all embodiments, and the various embodiments may be combined, when such incorporation and/or combination can be accomplished without undue experimentation. Therefore, the appended claims are to encompass within their scope all such changes and modifications as fall within the true spirit and scope of this invention.

What is claimed is:

1. A method of fabricating a coating for a medical device that controls the release of both a hydrophobic drug and a peptide comprising:
providing an implantable medical device;
providing a solvent;
providing a semi-crystalline or amorphous polymer
having a weight average molecular weight of not less than 50,000 Daltons;
having a glass transition temperature, when plasticized with water under physiological conditions, of not more than 45° C.;
and
having a solubility parameter between about 5 to about 25 (cal/cm³)^{1/2};
providing a peptide selected from the group consisting of RGD, cRGD, natriuretic peptide CNP, natriuretic peptide ANP, natriuretic peptide BNP, glycoprotein IIb/IIIb antagonists, Abciximax, anti-₃-integrin antibody F11, laminin derived SIKVAV, laminin derived YIGSR, KQAGDV, VAPG, and any combination thereof;
providing a hydrophobic drug with an aqueous solubility of not more than 1 mg/ml;
dissolving the peptide, the hydrophobic drug, and the polymer in the solvent to form a coating solution;
wherein the mass ratio of the peptide to the hydrophobic drug is from about 1:5 to about 5:1; and
wherein the mass ratio of the sum of the peptide and the hydrophobic drug to the polymer is from about 1:1 to about 1:7;
disposing the solution over a surface of the implantable medical device; and
removing the solvent.

2. The method of claim 1, wherein disposing the solution over the implantable medical device comprises spraying the solution onto the surface of the device.

3. The method of claim 1, wherein the mass ratio of the sum of the peptide and hydrophobic drug to the polymer is about 1:1 to about 1:5.

4. The method of claim 3, wherein the mass ratio of the sum of the peptide and hydrophobic drug to the polymer is about 1:3 to about 1:5.

5. The method of claim 1, wherein the polymer, when plasticized with water under physiological conditions, has a glass transition temperature not greater than 37° C.

6. The method of claim 1, wherein the polymer is a copolymer of ε-caprolactone and at least one monomer that would form an aliphatic polyester.

7. The method of claim 1, wherein the polymer is a co-polymer of two or more monomers wherein at least one monomer has a solubility parameter of greater than or equal to 12.9 (cal/cm³)^{1/2} and at least one monomer has a solubility parameter that differs from that of the drug by not more than 2.5 (cal/cm³)^{1/2}.

8. The method of claim 7, wherein the monomer(s) with a solubility parameter of greater than or equal to 12.9 (cal/cm³)^{1/2} comprise at least 25 mole % of the polymer and the monomer(s) with a solubility parameter that differs from that of the drug by not more than 2.5 (cal/cm³)^{1/2} comprise at least 25 mole % of the polymer.

9. The method of claim 1, wherein the polymer comprises a hydrophilic block selected from the group consisting of poly(ethylene glycol), poly(vinyl pyrrolidone), poly(vinyl alcohol), poly(vinyl acetate), and combinations thereof

10. The method of claim 1, wherein polymer comprises a poly(ester-amide) or an amphiphilic block copolymer.

11. The method of claim 10, wherein the poly(ester-amide) has the formula: 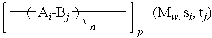
wherein:
i is an integer from 1 to 10, inclusive;
j is an integer from 1 to 10, inclusive;
xₙ is an integer from 1 to 100, inclusive;
p is an integer from 2 to about 4500;
M_{w} is from about 10,000 to about 1,000,000 Da;
sᵢ is a number from 0 to 0.5, inclusive;
tⱼ is a number from 0 to 0.5, inclusive;
with the proviso that
Σᵢsᵢ=Σⱼ tⱼ=0.5;
Σᵢs_{i>}0;
Σⱼtⱼ>0
each Aᵢ has the chemical structure: 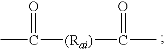
and
each Bⱼ has the chemical structure 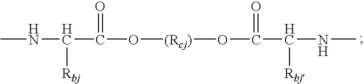
wherein:
each R_{bj}, and R_{bj′} is independently selected from the group consisting of hydrogen and (C1-C4)alkyl, wherein:
the alkyl group is optionally substituted with a moiety selected from the group consisting of —OH, —SH, —SeH, —C(O)OH, —NHC(NH)NH₂, 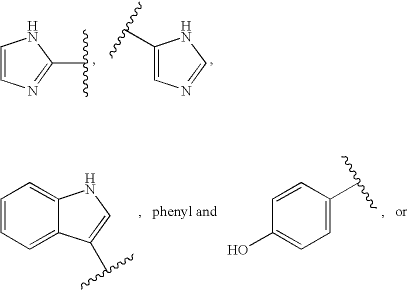
one or more of R_{bj} and R_{bj′} forms a bridge between the carbon to which it is attached and an adjacent nitrogen, the bridge comprising —CH₂CH₂CH₂—;
each Rₐᵢ and each R_{cj} is independently selected from the group consisting of (C1-C12)alkyl, (C2-C12)alkenyl, (C3-C8)cycloalkyl, —CH₂CH₂O)_{q}CH₂CH₂— wherein q is an integer from 1 to 10, inclusive, 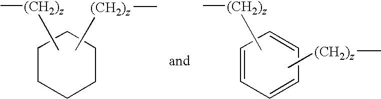
where z is 0, 1, or 2.

12. The method of claim 11, wherein for the polymer i=1 or 2, and j=2, and
each of Rₐ₁ is selected from the group consisting of
—(CH₂)₄—, —(CH₂)₅—, —(CH₂)₆—, —(CH₂)₇—, (CH₂)₈, —(CH₂)₁₀—, —(CH₂)₁₁, and —(CH₂)₁₂—;
each of R_{b1}, R_{b1′}, R_{b2} and R_{b2′} are the same, and are selected from the group consisting of —(CH₂)—(CH(CH₃)₂), —(CH₃), —CH(CH₃)₂, —(CH₂)₂—CO(NH₂), —CH(CH₃)—CH₂—CH₃, CH(OH)(CH₃), —CH₂—CO—(NH₂), —(CH₂)₄NH₃⁺, —(CH₂)₂—COO⁻, —(CH₂)₃NH—C(NH₂⁺)NH₂, —(CH₂)₂—S—(CH₃), and —(CH₂)—SH;
R_{c1} is selected from the group consisting of —(CH₂)₄—, —(CH₂)₅—, —(CH₂)₆—, CH₂)₇—, —(CH₂)₈—, —(—CH₂CH₂O—)₁(CH₂)₂—, —(—CH₂CH₂O—)₂(CH₂)₂—, and —(—CH₂CH₂O—)₃(CH₂)₂—;
R_{c2} is selected from the group consisting of 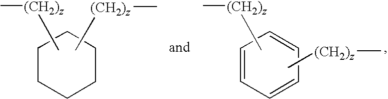
where z is 0, 1, or 2;
and t₁ is 0.125 to 0.375.

13. The method of claim 1, wherein the solvent is ethanol.

14. The method of claim 1, wherein the hydrophobic drug is selected from the group consisting of Biolimus A9, deforolimus, AP23572, tacrolimus, temsirolimus, pimecrolimus, zotarolimus, everolimus, 40-O-(3-hydroxypropyl)rapamycin, 40-O-[2-(2-hydroxy)ethoxy]ethyl-rapamycin, 40-O-tetrazole-rapamycin, 40-O-tetrazolylrapamycin, 40-epi-(N1-tetrazole)-rapamycin, paclitaxel, and combinations thereof.

15. A method of fabricating a coating for a medical device that controls the release of both a hydrophobic drug and a peptide comprising:
providing an implantable medical device;
providing a first solvent;
providing a first polymer, a semi-crystalline or amorphous polymer,
having a number average molecular weight of not less than 50,000 Daltons;
having a glass transition temperature, when plasticized with water under physiological conditions, of not more than 45° C.;
and
having a solubility parameter between about 5 to about 25 (cal/cm³)^{1/2};
providing a peptide selected from the group consisting of RGD, cRGD, natriuretic peptide CNP, natriuretic peptide ANP, natriuretic peptide BNP, glycoprotein IIb/IIIb antagonists, Abciximax, anti-₃-integrin antibody F11, laminin derived SIKVAV, laminin derived YIGSR, KQAGDV, VAPG, and any combination thereof;
providing a hydrophobic drug, that is different from the peptide, with an aqueous solubility of not more than 1 mg/ml;
dissolving the peptide and the first polymer, and optionally the hydrophobic drug, in the first solvent to form a first coating solution;
wherein the mass ratio of the peptide to polymer, or the sum of peptide and hydrophobic drug to polymer if the hydrophobic drug is added, is from 3:1 to 1:10;
disposing the first coating solution over a surface of the implantable medical device;
and removing the solvent;
optionally forming an optional intermediate coating layer by:
providing an intermediate layer solvent, which may be the same as or different from the first solvent;
dissolving the hydrophobic drug in the intermediate layer solvent to form an intermediate layer coating solution; and
disposing the intermediate layer coating solution over a coated surface of the implantable medical device;
removing the solvent;
providing a second solvent, which may be the same as or different from either the first solvent and/or the optional intermediate layer solvent;
providing a second semi-crystalline or amorphous polymer,
having a number average molecular weight of not less than 50,000 Daltons;
having a glass transition temperature, when plasticized with water under physiological conditions, of not more than 45° C.; and
having a solubility parameter that differs from the solubility parameter of the first polymer by not more than 10 (cal/cm³)^{1/2};
dissolving the second polymer, and optionally the hydrophobic drug, in the second solvent to form a second coating solution;
wherein if the second coating solution comprises the hydrophobic drug, the mass ratio of the drug to polymer is from 1:1 to 1:5;
disposing the second coating solution over a coated surface of the implantable medical device; and
removing the solvent;
wherein at least one of the first, second, or optional intermediate coating solutions comprises the hydrophobic drug.

16. The method of claim 15, wherein disposing the first coating solution, intermediate coating solution and the second coating solution comprises spraying the solution on the surface or a coated surface of the device.

17. The method of claim 15, wherein the mass ratio of the sum of the peptide and hydrophobic drug to the first polymer or mass ratio of peptide to polymer is about 1:4 to about 1:8.

18. The method of claim 15, wherein there is an intermediate coating layer comprising the hydrophobic drug.

19. The method of claim 15, wherein second coating solution comprises the hydrophobic drug.

20. The method of claim 15, wherein the second polymer, when plasticized with water, has a glass transition temperature not greater than 37° C.

21. The method of claim 15, wherein the first polymer, when plasticized with water, has a glass transition temperature not greater than 37° C.

22. The method of claim 15, wherein the first polymer is an amphiphilic block copolymer comprising a polar block.

23. The method of claim 22, wherein the polar block is selected from the group consisting of poly(urethane), poly(HEMA-block-MMA), poly(HEMA-block-HPMA), poly(HPMA-GFLG), poly(butyl methacrylate-co-ethylene glycol acrylate) (poly(BMA-block-PEGA)) poly(MOEMA-block-HEMA), and any combination thereof.

24. The method of claim 22, wherein the polar block comprises no less than 25 mole % of the polymer and no more than 75 mole % of the polymer.

25. The method of claim 15, wherein the second polymer has a solubility parameter about equal to or less than 12 (cal/cm³)^{1/2}.
